(19) United States
(12) Patent Application Publication
(10) Pub. No.: US 2003/0077814 A1
(43) Pub. Date: Apr. 24, 2003

(54) Novel lactic acid bacteria

(76) Inventor: Jong Suk Oh, Kwangju-City (KR)

Correspondence Address: FROMMER LAWRENCE & HAUG, 745 FIFTH AVENUE- 10TH FL., NEW YORK, NY, 10151, US

(21) Appl. No.: 10/122,543

(22) Filed: Apr. 15, 2002

Related U.S. Application Data
(62) Division of application No. 09/492,991, filed on Jan. 27, 2000, now abandoned.
Application No. 09/492,991 is a continuation-in-part of application No. 09/014,436, filed on Jan. 27, 1998, now Pat. No. 6,036,952.

(30) Foreign Application Priority Data
Aug. 7, 1997 (KR) 1997/37819
Jan. 7, 1998 (KR) 1998/213

(51) Int. Cl. C12N 1/20

(52) U.S. Cl. 435/252.9

(57) ABSTRACT

Enterococcus spp. 1357, Lactobacillus spp. V20 and Lactococcus spp. 1370, and H₂O₂-producing Streptococci have a potent and lasting inhibitory activity on the production of water-insoluble glucan (mutan) and dental plaque in human mouth, or the growth of anaerobic bacteria causing gingivitis, periodontitis, and accompanied halitosis.

### BACKGROUND OF THE INVENTION

1. Field of the Invention

The present invention relates to some novel lactic acid bacteria which inhibit the production of dental plaque in human mouths. More specifically, the production of water-insoluble glucan (mutan), a major component of dental plaque, which is produced by bacteria normally inhibiting in human mouths, can be inhibited by the novel bacteria. Oral anaerobic bacteria causing gingivitis, periodontitis, and accompanied halitosis (malodor) can be inhibited by the novel bacteria, too. These lactic acid bacteria belong to Enterococcus spp., Lactobacillus spp., Lactococcus spp., and Stretococcus spp. which inhibit the production of water-insoluble glucan or antagonize against the bacteria playing a role in forming water-insoluble glucan, or inhibit the growth of anaerobic bacteria causing gingivitis and periodontitis.

2. Description of the Prior Art

Lactic acid bacteria generally ferment carbohydrates to lactic acid. Lactic acid bacteria live in the oral cavities and the alimentary tracts of men and animals and are utilized for the manufacture of fermentative foods, such as yogurt, cheese, etc. In addition, they are used for the production of biologically active materials, such as medicines. Representatives of these lactic acid-producing bacteria are Streptococcus thermophilus, Enterococcus faecalis, Enterococcus durans, Lactococcus lactis, Lactobacillus acidophilus, Lactobacillus bulgaricus, Lactobacillus casei, and Lactobacillus plantarum. As inhabitants in the entrails of men and animals, these Gram-positive lactic acid bacteria are known to play an important role in maintaining the entrails healthy by the production of lactic acid and antibacterial materials which inhibit the growth of pathogenic bacteria.

The most important component of dental plaque is glucan. Glucan is either water-soluble glucan, dextran having 1,6-α linkage as a predominant linkage, or water-insoluble glucan (mutan) having 1,3-α linkage as a predominant linkage. The solubility in water is inversely proportional to the number of 1,3-α linkage. Therefore, water-insoluble glucan (mutan) serves as a main matrix of dental plaque. Dextranase (α-1,6 glucan hydrolase) which digests dextran, was tested as to its ability to prevent dental plaque. But the effectiveness value of dextranse to prevent dental plaque was questionable (Essential Dental Microbiology: Appleton & Lange, Norwalk, San Mateo, p.337, 1991), because dextranase can not digest mutan, the main matrix of dental plaque. Mutanase (endo-α-1,3-glucanase) which decomposes mutan was found to have some effect on the digestion of dental plaque. The decomposition effect of the mutanase on dental plaque, however, was trivial and it took too much time to express its effectivity. Therefore, these enzymes were found to have an insignificant effect on dental plaque formation in human oral cavity.

With regard to dental plaque prevention with lactic acid bacteria, European patent publication number 0-524-732-A2 disclosed the use of Streptococcus salivarius which was capable of extracellular production of dextranase. But its effect on preventing dental plaque is questionable because dextranase can not digest mutan, the main matrix of dental plaque. Streptococcus salivarius is not used as the starter bacteria fermenting milk.

### SUMMARY OF THE INVENTION

As a consequence of intensive and thorough researches on the inhibitory activity of lactic acid bacteria against the production of water-insoluble glucan or dental plaque and the growth of anaerobic bacteria, the present study has been based on the presumption that some lactic acid bacteria inhabiting in human mouths may be able to inhibit the production of water-insoluble glucan or dental plaque or the growth of anaerobic bacteria causing gingivitis and periodontitis. Through many clinical experiments, these novel strains have been found and proved to have the ability to inhibit the production of water-insoluble glucan or dental plaque significantly, and to inhibit the growth of anaerobic bacteria causing gingivitis and periodontitis. They was named Enterococcus spp. 1357, Lactobacillus spp. V20, and Lactococcus spp. 1370, respectively. They are now deposited in the Korean Collection for Type Cultures, Korean Research Institute of Bioscience and Biotechnology, on Jul. 30, and Dec. 11, 1997 (deposition No. KCTC 0360BP for Enterococcus spp. 1357, KCTC 0361BP for Lactobacillus spp. V20, KCTC 0415BP for Lactococcus spp. 1370).

Glucan is either water-soluble or water-insoluble (mutan), each being synthesized from sucrose by the glucosyltransferase secreted from Streptococcus mutans, the most important bacteria among dental plaque-forming bacteria. However, only the water-insoluble glucan, mutan, is the main matrix of dental plaque.

Enterococcus spp. 1357, Lactobacillus spp. V20, and H₂O₂-producing Streptococci such as Streptococcus oralis, Streptococcus mitior, Streptococcus mitis, and Streptococcus sanguis (Bergey's Manual of Systematic Bacteriology vol. 2: Williams & Wilkins, Baltimore, London, Los Angeles, Sydney, 1986) have a growth-inhibitory activity on Streptococcus mutans. When Streptococcus mutans was cultured with Enterococcus spp. 1357, Lactobacillus spp. V20, or Streptococcus oralis (ATCC 35037) as a representative of H₂O₂-producing Streptococci in the broth, the colony-forming number of Streptococcus mutans was decreased to about one hundredth compared with that of Streptococcus mutans alone. The production of water-insoluble glucan or dental plaque was also suppressed significantly due to inhibition of Streptococcus mutans.

When high molecular weight dextran was added into the culture broth of Streptococcus mutans, the glucosyltransferase binding to water-insoluble glucan was interfered, and then the following synthesis of water-insoluble glucan was inhibited (Shigeyuki H., et al., Journal of General Microbiology, 116:51, 1980). Lactococcus spp. 1370 produced the large amounts of water-soluble glucan; when Streptococcus mutans was incubated with this Lactococcus spp. 1370, the synthesis of water-insoluble glucan was suppressed.

Generally, dental plaque, adherent to the surface of teeth, provides a suitable habitat at which Streptococcus mutans as well as other bacteria proliferate and causes the dental caries formed, and it was an object of the present study to find novel bacteria which could inhibit the production of water-insoluble glucan or dental plaque by Streptococcus mutans in the mouth.

Anaerobic bacteria occur in high proportions in periodontitis as well as gingivitis of oral cavities. The proportions of anaerobic bacteria increase significantly (above 90% of microflora in periodontitis lesion) with increasing severity of the diseases. Predominant anaerobic bacteria causing gingivitis are Prevotella intermedia and Fusobacterium nucleatum. Predominant anaerobic bacteria causing periodontitis include Porphyromonas gingivalis, Actinobacillus actinomycetemcomitans, Prevotella intermedia, and Fusobacterium nucleatum (Contemporary Oral Microbiology and Immunology, Mosby-Year Book, St. Louis, 1992). These anaerobic bacteria produce malodorous components such as volatile sulfur compounds in mouth. The predominant volatile sulfur compounds are hydrogen sulfide from L-cysteine and methyl mercaptan from L-methionine (Persson, S., Oral Microbiol Immunol, 7:378, 1992). Lactobacillus spp. V20 and H₂O₂-producing Streptococci such as Streptococcus oralis, Streptococcus mitior, Streptococcus mitis, and Streptococcus sanguis produce hydrogen peroxide inhibiting the growth of anaerobic bacteria causing gingivitis and periodontitis, so improving and preventing the lesions, and decreasing the accompanied halitosis. The hydrogen peroxide is an oxidizing agent inactivating enzymes by converting functional —SH groups to the oxidized S—S form and is used as a disinfectant against bacteria, especially anaerobics.

Another object of this research was to provide foods or beverages employing the lactic acid bacteria. When the foods containing the lactic acid bacteria capable of directly inhibiting the production of water-insoluble glucan or having the growth-inhibitory activity on the microorganisms which contribute to the formation of dental plaque are eaten, the lactic acid bacteria naturally suppress the formation of dental plaque and consequently prevent dental caries formation. And when the foods contain the lactic acid bacteria capable of inhibiting the growth of anaerobic bacteria causing gingivitis and periodontitis, the lactic acid bacteria naturally improve and prevent gingivititis, periodontitis, and accompanied halitosis.

### BRIEF DESCRIPTION OF THE DRAWING

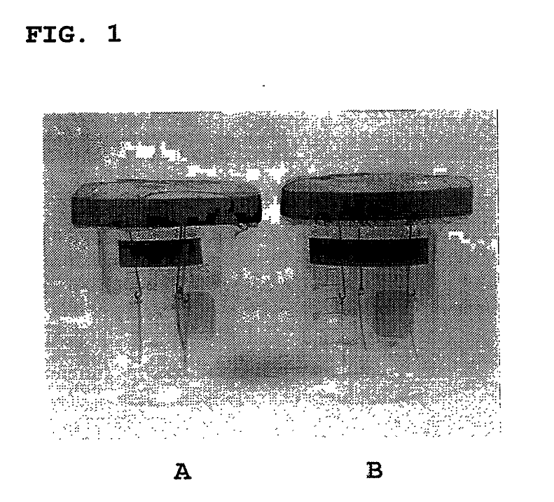

Above and other objects and aspects of the invention will become apparent from the following description of embodiment with reference to the accompanying drawing which shows the inhibitory effect of the novel strain on the production of artificial dental plaque on orthodontic wires (FIG. 1).

### DETAILED DESCRIPTION OF PROCESS TO THE INVENTION

Lactic acid bacteria were taken from human bodies, streaked on Brain Heart Infusion agar, and cultured at 37° C. Thereafter, separated bacterial colonies were tested whether they could suppress the production of the water-insoluble glucan that Streptococcus mutans (Ingbritt strain) produced. In a cuvette, 3 mL of a Brain Heart Infusion medium supplemented with 0.5% yeast extract and 5% sucrose was inoculated with 0.1 mL of Streptococcus mutans and 0.1 mL of a culture broth of the separated bacteria. As a control, Streptococcus mutans was inoculated alone in a Brain Heart Infusion medium containing yeast extract and sucrose. The cuvette was placed at an angle of 30° to the horizontal plane of an incubator and incubated for 1 day at 37° C., in order for the Streptococcus mutans to produce water-insoluble glucan. After removing the culture broth, the cuvette was washed with 4 mL of distilled water and then, filled with 3 mL of distilled water. Its absorbance (OD) at 550 nm was measured by a spectrophotometer. Because the OD value was proportional to the water-insoluble glucan produced, the bacteria which brought about a significantly lower OD value compared with the control, were isolated as dental plaque-inhibitory strains.

To evaluate the capability producing hydrogen peroxide, the isolated strains were streaked on the Brain Heart Infusion agar containing 0.25mg/mL 3,3′,5,5′-tetramethylbenzidine dihydrochloride and 0.01 mg/mL horseradish peroxidase and incubated in anaerobic incubator for 48 hours. Lactobacillus spp. V20 formed the blue colonies, indicating that Lactobacillus spp. V20 had the capability to produce hydrogen peroxide.

The microbiological properties of the isolated strains, such as morphological and physiological properties (Table 1), and sugar catabolytic ability (Table 1) were investigated.

TABLE 1
Morphological and Physiological Properties

| | Isolated Bacterial Strains | | |
|---|---|---|---|
| | Enterococcus | Lactobacillus | Lactobacillus |
| Properties | spp. 1357 | spp. V20 | spp. 1370 |
| Morphology | coccus, | bacillus, | coccus, chain |
| | chain | chain | |
| Gram stain | positive | positive | positive |
| Spore forming | − | − | − |
| Catalase | − | − | − |
| Activity | | | |
| Culture | + | − | + |
| Temp. 10° C. | | | |
| Culture | + | + | − |
| Temp. 45° C. | | | |
| pH 9.6 | + | − | − |
| 40% bile acid | + | − | + |
| 6.5% NaCl | + | − | − |
| Growth on MRS | − | + | − |
| medium | | | |
| Acetoin | + | | + |
| Production | | | |
| Hippurate | + | | − |
| Hydrolysis | | | |
| Pyrrolidonylary | + | | − |
| amidase | | | |
| α- | − | | − |
| Galactosidase | | | |
| β- | − | | − |
| Glucuronidase | | | |
| β- | + | | − |
| Galactosidase | | | |
| Alkaline | − | | − |
| phosphatase | | | |
| Leucine | + | | + |
| arylamidase | | | |
| Arginine | + | | − |
| dihydrolase | | | |

TABLE 2
Catalytic Activities on Sugars

| | Isolated Bacterial Strains | | |
|---|---|---|---|
| | Enterococcus | Lactobacillus | Lactococcus |
| Carbohydrate | spp. 1357 | spp. V20 | spp. 1370 |
| Arabinose | − | − | − |
| Amygdalin | + | − | − |
| Cellobiose | + | + | + |
| Esculin | + | + | − |
| Fructose | + | + | + |
| Galactose | + | + | + |
| Glucose | + | + | + |
| Lactose | + | + | + |
| Maltose | + | + | + |
| Mannitol | − | − | + |
| Mannose | + | + | + |
| Melezitose | − | − | − |
| Raffinose | − | − | − |
| Rhamnose | − | − | − |
| Salicin | + | − | − |
| Sorbitol | − | − | − |
| Trehalose | + | + | + |
| Inulin | − | | − |
| Starch | + | | + |
| Glycogen | − | | − |

As mentioned above, the novel lactic acid bacteria being able to inhibit the production or dental plaque were isolated and assayed in comparison with the control in vitro. Further, the novel properties were tested in vivo, that is, the isolated novel bacteria were applied to the human oral cavity.

### EXAMPLE I

### Inhibition of the Production of Water-Insoluble Glucan in Disposable Cuvette

An equal amount of M17 medium was mixed with MRS medium and supplemented with 0.5% yeast extract, 5% sucrose and 0.1M TES (pH 8.0). Three milliliters of the constituted medium were transferred to a disposable cuvette which was, then, inoculated with 75 μl of Streptococcus mutans overnight culture. The cuvette was placed at an angle of 30° to the horizontal plane in an incubator and cultured at 30° C. for 1 day. The content was removed and then, the cuvette was washed with 3 mL of distilled water. Thereafter, the cuvette was filled with 4 mL of distilled water and the absorbency at 550 nm was measured by a spectrophotometer. This measurement was repeated three times and the average value was obtained (Table 3).

TABLE 3
Inhibitory Effect of Lactic Acid Bacteria on the Production
of Water-Insoluble Glucan

| Samples | Test Bacterial Strains | OD (550 nm) |
|---|---|---|
| Control I | Str. mutans | 2.122 |
| Control II | Streptococcus thermophilus + | 2.325 |
| | Str. mutans | |
| Group I | Enterococcus spp. 1357 | 0.434 |
| Group II | Enterococcus spp. 1357 + | 0.713 |
| | Str. mutans | |
| Group III | Lactobacillus spp. V20 | 0.506 |
| Group IV | Lactobacillus spp. V20 + | 1.154 |
| | Str. mutans | |
| Group V | Lactococcus spp. 1370 | 0.576 |
| Group VI | Lactococcus spp. 1370 + | 1.020 |
| | Str. mutans | |
| Group VII | Streptococcus oralis | 0.511 |
| Group VIII | Streptococcus oralis + | 0.980 |
| | Str. mutans | |

As shown in Table 3, the optical densities of Control group I and Control group II were 2.122 and 2.325 in absorbency at 550 nm whereas those of Test group II, Test group IV, Test group VI, and Test group VIII were 0.713, 1.154, 1.020, and 0.980, respectively. This reduction in the absorbency meant that these bacteria inhibited the Streptococcus mutans production of water-insoluble glucan.

### EXAMPLE II

### Artificial Dental Plaque Formation on Orthodontic Wire

An equal amount of M17 medium was mixed with MRS medium and supplemented with 0.5% yeast extract, 5% sucrose, and 0.1M TES (pH 8.0). One hundred and fifty milliliters of the constituted medium were poured into beaker. 0.016 inch stainless steel orthodontic wires (45 mg) were immersed in the medium. Streptococcus mutans was inoculated at the concentration of 2.5×10⁶ per mL of the medium. Thereafter, the lactic acid bacterial strains were inoculated in the medium at the concentration of 5 times higher than that of Streptococcus mutans and incubated in a CO₂ incubator at 37° C. for 6.5 hours with shaking. Only water-insoluble glucan or plaque was attached on the wires (McCabe, R. M., et. Al., Archs oral Boil., 12:1653, 1967). The wires were transferred to fresh beakers and photographed (FIG. 1). FIG. 1(A) is a photograph of the culture of Streptococcus mutans alone while FIG. 1(B) is that of the co-culture of Streptococcus mutans and Lactococcus spp. 1370.

The weights of the artificial plaques formed on the wires were measured and the results are shown in Table 4.

TABLE 4
Inhibitory Activity of Lactic Acid Bacteria
on the Formation of Artificial Plaque

| | | Weight of |
|---|---|---|
| Samples | Test Bacterial Strains | Produced Plaque |
| Control I | Str. mutans | 75.4 mg |
| Control II | Streptococcus thermophilus + | 92.3 mg |
| | Str. mutans | |
| Group I | Enterococcus spp. 1357 + | 0.0 mg |
| | Str. mutans | |
| Group II | Lactobacillus spp. V20 + | 30.9 mg |
| | Str. mutans | |
| Group III | Lactococcus spp. 1370 + | 0.0 mg |
| | Str. mutans | |
| Group IV | Streptococcus oralis + | 0.0 mg |
| | Str. mutans | |

In the control group I and the control group II, an artificial plaque of 75.4 mg and 92.3 mg was formed while no artificial plaque was formed in Test group I, Test group III and Test group IV. In Test group II, the plaque weight was reduced to 30.9 mg. Thus, it is clearly shown that these bacteria have a potent inhibitory activity on the production of dental plaque by Streptococcus mutans.

### EXAMPLE III

Reduction of the Dental Plaque Index in Human Mouths

In order to evaluate the reduction effect of the novel lactic acid bacterial strains on plaque index in human mouths, experiments were performed in thirty-eight persons to achieve the plaque scores by Quigley and Hein Plaque Index (Harper, D. S., et. Al., J Periodontol, 61:352, 1990).

Thirty-eight young adults, 22 to 26 years of age, volunteered to participate in this study. All volunteers were received thorough oral prophylaxis, and suspended all oral hygiene. Volunteers ate and drank as usual, but stopped brush washing. Baseline plaque scores were assessed at 24 hours after receiving oral prophylaxis. Plaque scores were performed by Quigley and Hein Plaque Index after disclosing all plaque except third molars. The volunteers were randomly assigned to two groups (each nineteen persons), group I mouthrinsing with Lactococcus spp. 1370 while group II mouthrinsing with Lactobacillus spp. V20. Test bacterial suspensions were prepared by incubating either Lactococcus spp. 1370 or Lactobacillus spp. V20 in milk for 24 hours. Volunteers rinsed immediately once after oral prophylaxis and twice after meals with 20 mL of Lactococcus spp. 1370 or Lactobacillus spp. V20 culture in milk (10⁹ CFU/mL) for 2 minutes. Plaque scores were again assessed at 24 hours after receiving oral prophylaxis. The plaque scores of total teeth except third molars were averaged and statistically analyzed in each group. The results indicated that plaque index reduction of 0.97 in the group I and 0.55 in the group II at 24 hours after receiving oral prophylaxis (Table 5). The reductions of plaque index were statistically significant (p<0.05), i.e. Lactococcus sp. 1370 and Lactobacillus sp. V20 reduced plaque formation in the oral cavity significantly.

TABLE 5
Reduction of the Plaque Index by Bacterial Mouthrinse

| | Mean | Mean Plaque Score | |
|---|---|---|---|
| | Baseline | afer Bacterial | |
| Used Bacteria | Plaque Score | Mouthrinse | Difference |
| Lactococcus | 2.17 | 1.20 | −0.97* |
| spp. 1370 | | | |
| Lactobacillus | 2.15 | 1.60 | −0.55* |
| spp. V20 | | | |

*p < 0.01 by paired t test

### EXAMPLE IV

### Inhibition of the Growth of Anaerobic Bacteria in Mixed Culture

Lactobacillus spp. V20 was cultured in MRS media for 24 hours. Prophyromonas gingivalis, Prevotella intermedia, and Fusobacterium nucleatum were cultured in the anaerobic bacteria culture broth containing Brain Heart Infusion media 18.5 g, yeast extract 5.0 g, hemin solution 10 mL (dissolved 50 mg hemin in 1 N sodium hydroxy solution 1 mL and added with distilled water 100 mL), and vitamin K solution 0.2 mL (vitamin K solution 0.15 mL mixed with 95% ethanol 30 mL) per liter in anaerobic incubator for 36 hours.

Actinobacillus actinomycetemcomitans was cultured in the TSBV media containing Tryptic soy broth 30 g, yeast extract 1.0 g, horse serum 100 mL, bacitracin 75 mg, and vancomycin 5 mg per liter in anaerobic incubator for 36 hours. Culture suspension 0.1 mL of Lactobacillus spp. V20 and each anaerobic bacterium at the concentration of 1.4×10⁸ per mL were inoculated singly or in combination in the media containing anaerobic bacteria culture broth or TSBV media 3.7 mL mixed with MRS broth 0.3 mL, and cultured in anaerobic incubator for 36 hours. The culture suspension was diluted and inoculated on MRS agar, anaerobic bacteria culture agar containing 3% sheep blood or TSBV agar. At 72 hours after culturing, the number of colonies was counted. The colony-forming units of Lactobacillus spp. V20 and each anaerobic bacterium were increased after being cultured singly, whereas the colony of each anaerobic bacterium was not found after being cultured in combination with Lactobacillus spp. V20. When anaerobic bacteria was cultured with Lactobacillus casei which did not produce hydrogen peroxide, the colony-forming units were not decreased significantly (Table 5). When Streptococcus oralis (ATCC 35037) as a representative of H₂O₂-producing Streptococci was cultured with each anaerobic bacterium by the above mentioned method, anaerobic bacteria did not form a colony on the media.

TABLE 6
Colony-forming Units after Culturing

| | Colony-forming | Colony-forming |
|---|---|---|
| | units of | units of |
| Inoculated | Lactobacillus | anaerobe after |
| bacteria | after culture(/mL) | culture (/mL) |
| Lactobacillus spp. | 8.2 × 10⁸ | |
| V20 | | |
| Lactobacillus casei | 9.0 × 10⁸ | |
| Porphyromonas | | 1.9 × 10⁸ |
| gingivalis | | |
| Actinobacillus | | 2.0 × 10⁸ |
| actinomycetemcomitans | | |
| Prevotella intermedia | | 1.8 × 10⁸ |
| Fusobacterium | | 1.8 × 10⁸ |
| nucleatum | | |
| Lactobacillus spp. | 8.0 × 10⁸ | 0 |
| V20 + Porphyromonas | | |
| gingivalis | | |
| Lactobacillus spp. | 8.8 × 10⁸ | 0 |
| V20 + Actinobacillus | | |
| actinomycetemcomitans | | |
| Lactobacillus spp. | 8.9 × 10⁸ | 0 |
| V20 + Prevotella | | |
| intermedia | | |
| Lactobacillus spp. | 7.8 × 10⁸ | 0 |
| V20 + Fusobacterium | | |
| nucleatum | | |
| Lactobacillus casei + | 9.1 × 10⁸ | 1.5 × 10⁸ |
| Porphyromonas | | |
| gingivalis | | |
| Lactobacillus casei + | 9.3 × 10⁸ | 1.8 × 10⁸ |
| Actinobacillus | | |
| actinomycetemcomitans | | |
| Lactobacillus casei + | 8.9 × 10⁸ | 1.6 × 10⁸ |
| Prevotella intermedia | | |
| Lactobacillus casei + | 9.8 × 10⁸ | 1.6 × 10⁸ |
| Fusobacterium | | |
| nucleatum | | |

Hereafter were presented the examples in which the novel lactic acid bacteria were practically applied.

### Use Example I

Yogurt

A broth culture containing the novel lactic acid bacterial strains was added at an amount of 0.1 vol. percent to the food just before fermentation and subjected to fermentation along with the existing bacteria to produce yogurt foods. The resulting yogurt foods were tasted by 10 panelists. They noted no different flavor between the test samples and the commercially available foods (controls).

Before a sealing step in the manufacture procedure, the lactic acid bacterial strains were added at an amount of 0.2 vol. percent. A response that these test samples thus obtained were not different from the control foods in taste was drawn from 10 panelists who took part in the tasting tests.

### Use Example II

Butter

Before a packaging step, butter foods which were manufactured by a typical procedure were added with 0.2 wt. percent of the freeze-dried lactic acid bacterial strains. These butter foods thus obtained were given as taste samples.

### Use Example III

Cheese

Before a packaging step, cheese foods which were manufactured by a typical procedure were added with 0.2 wt. percent of the freeze-dried lactic acid bacterial strains. These cheese foods thus obtained were given as taste samples.

### Use Example IV

Freeze-dried Lactic Acid Bacteria

The novel lactic acid bacteria were cultured and freeze-dried (lyophilized). These freeze-dried bacteria in capsule, tablet, and small package could be taken singly or with other bacteria or materials. These freeze-dried products thus obtained were given taste samples.

Accordingly, the lactic acid bacterial strains were applied for various foods, including gum, shortening, ice cream, margarine, kimchi, etc.

From the examples above, it is apparent that the novel lactic acid strains have a potent and lasting inhibitory effect on the production of water-insoluble glucan or dental plaque in human mouth, or on the growth of anaerobic bacteria causing gingivitis, periodontitis, and accompanied halitosis.

In addition, the novel strains were found to be able to be applied for various foods as well as directly to the teeth.

Many modifications and variations of the present invention are possible in the light of the above techniques.

What is claimed is:

1. Lactic acid bacteria which inhibit the production of water-insoluble glucan (mutan) or dental plaque by affecting the enzyme glucosyltransferase, or have a growth-inhibitory effect on dental plaque-forming bacteria in the oral cavity.

2. Lactic acid bacteria in accordance with claim 1 are Enterococcus spp. 1357 (KCTC 0360BP), Lactobacillus spp. V20 (KCTC 0361BP), and Lactococcus spp. 1370 (KCTC 0415BP).

3. Lactic acid bacteria which inhibit the growth of anaerobic bacteria causing gingivitis, periodontitis, and accompanied halitosis (malodor).

4. Lactic acid bacteria which inhibit hydrogen peroxide (H₂O₂) to inhibit the growth of anaerobic bacteria in accordance with claim 3.

5. Lactic acid bacteria in accordance with claim 4 are Lactobacillus spp. V20 (KCTC 0361BP) and Streptococcus oralis (ATCC 35037).

6. Food which can inhibit either the production of water-insoluble glucan or dental plaque by affecting the enzyme glucosyltransferase or the growth of dental plaque-forming bacteria, containing the lactic acid bacterial strains of claim 1 alone or in mixture.

7. Food which can inhibit the growth of anaerobic bacteria causing gingivitis, periodontitis, and accompanied halitosis, containing the lactic acid bacterial strains of claim 4 alone or in mixture.

8. Food in accordance with claim 6 or 7 can be yogurt, butter, cheese, ice cream, gum, shortening, margarine, or kimchi.

9. Freeze-dried form of the lactic acid bacteria of the claim 1 or 4 in the form of capsule, tablet or small package.
